(19) United States
(12) Patent Application Publication
(10) Pub. No.: US 2024/0115133 A1
(43) Pub. Date: Apr. 11, 2024

(54) EEG SIGNAL CAPTURE DEVICE COMPRISING A PLURALITY OF SENSORS DISTRIBUTED OVER THE HEAD

(71) Applicant: NAIXES, AIX-EN-PROVENCE (FR)

(72) Inventor: Bruno CHARRAT, AIX-EN-PROVENCE (FR)

(21) Appl. No.: 18/481,370

(22) Filed: Oct. 5, 2023

(30) Foreign Application Priority Data
Oct. 7, 2022 (FR) 2210279

(51) Int. Cl. A61B 5/00; A61B 5/257; A61B 5/291; A61B 5/369

(52) U.S. Cl. CPC A61B 5/0006; A61B 5/257; A61B 5/291; A61B 5/369; A61B 5/6814; A61B 2560/0214; A61B 2562/0209; A61B 2562/164

(57) ABSTRACT

A device for capturing EEG signals comprises a flexible wireless sensor configured to be adhesively attached at an EEG measurement point of a user's head, and configured to be remotely powered and to transmit, via a first radio frequency link, EEG data corresponding to samples of a measured EEG signal; a base station located near the sensor, configured to remotely power the sensor; and a terminal configured to receive transmitted EEG data.

### CROSS-REFERENCE TO RELATED APPLICATION

This application is based on and claims priority under 35 U.S.C. 119 to French Patent Application No. FR2210279 filed on Oct. 7, 2022, the disclosure of which is herein incorporated by reference in its entirety.

### TECHNICAL FIELD

The disclosure relates to the measurement of electroencephalograms (EEG) to determine brain states in certain human activities requiring a high level of concentration.

### BACKGROUND

Certain EEG signals can be used to quantify brain states such as the level of relaxation, stress, attention, fatigue, mental load, and even preparation for muscle action.

In general, according to the International 10-20 System, a complete EEG can be performed using about twenty electrodes in electrical contact with specific points on the skull, on which voltages are measured. A reference electrode is also provided for the voltage measurements.

To determine certain brain states, it is sufficient to detect EEG signals at the two forehead points (called Fp1 and Fp2) and one point behind each ear (A1, A2).

The electrodes are generally arranged in devices worn on the heads of users, whose configuration makes them poorly suited for real-world use in the field with a moving person, outside of an adapted experimental environment.

### SUMMARY

A device for capturing EEG signals is generally provided, comprising a flexible wireless sensor adhesively attached at an EEG measurement point of a user's head, configured to be remotely powered and to transmit, via a first radio frequency link, EEG data corresponding to samples of a measured EEG signal; a base station located near the sensor, configured to remotely power the sensor; and a terminal configured to receive transmitted EEG data.

The base station may be configured to receive EEG data transmitted by the sensor and retransmit the EEG data via a second radio frequency link to the terminal.

The terminal may be configured to receive EEG data directly from the sensor.

The sensor may comprise a flexible substrate; on a first face of the substrate, two conductive zones configured to electrically contact the skin and form two electrodes of an EEG signal detector; a conductive track on one side of the substrate, forming a remote power supply antenna; a control circuit arranged on a second side of the substrate, connected by conductive tracks of the substrate to the EEG signal detector and to the remote power supply antenna, the control circuit being configured to extract its power supply from the signal received by the remote power supply antenna; and digitize the signal from the EEG signal detector and transmit the corresponding EEG data via the first radio-frequency link.

One of the electrode conductor zones may be connected to a ground of the control circuit and the other electrode conductor zone may form a segment of the antenna track, the control circuit being configured to extract the power supply and the EEG signal from the antenna signal.

The base station may be attached to a device worn on the user's head.

The base station may be configured to synchronize the sampling of EEG signals of the wireless sensor.

The device may comprise a plurality of wireless sensors, the wireless sensors cooperating so that a master wireless sensor synchronizes the sampling of EEG signals of all the wireless sensors.

The synchronization may implement synchronization pulses transmitted by radio frequency in a frequency band distinct from that used for EEG data transmission and remote powering.

The base station may be configured to suspend the remote power supply at periodic intervals and the wireless sensor be configured to acquire at least one sample of the EEG signal as soon as the power supply to the wireless sensor becomes sufficient after a suspension.

The base station may be configured to interrupt the remote power supply at periodic intervals brief enough to not interrupt power to the wireless sensor, and the wireless sensor be configured to synchronize EEG signal sampling with the remote power supply interruption intervals.

The device may comprise a plurality of wireless sensors distributed over the skulls of a plurality of users, and a plurality of base stations arranged in respective devices carried on the heads of the users, each base station being configured to relay to the remote terminal: EEG data received from sensors in its remote power supply range, EEG data received from sensors outside its remote power-supply range, or EEG data received from another base station.

The EEG data may be transmitted by the sensor or base station via a WiFi or Bluetooth link.

### BRIEF DESCRIPTION OF DRAWINGS

Embodiments will be described in the following description, made by way of non-limiting example in relation to the accompanying drawings, among which:

FIG. 1 illustrates an embodiment of an EEG signal capture device comprising a plurality of wireless sensors distributed over the head;

FIGS. 2A and 2B represent a top view and a cross-sectional view of an embodiment of a wireless and batteryless EEG sensor;

FIG. 3 represents a block diagram of electronic architecture of an EEG signal capture device using a wireless sensor according to FIGS. 2A and 2B;

FIG. 4 represents a top view of another embodiment of a wireless EEG sensor;

FIG. 5 represents an analog subsystem of a control circuit of the sensor of FIG. 4;

FIG. 6 illustrates an implementation of several wireless sensors distributed over several people;

FIG. 7 represents a block diagram of electronic architecture of an EEG signal capture device implementing a first mode of synchronization between several wireless EEG sensors having an adapted control circuit;

FIG. 8 is a timing diagram illustrating another mode of synchronization of several wireless EEG sensors; and

FIG. 9 is a timing diagram illustrating another mode of synchronization of several wireless EEG sensors.

### DETAILED DESCRIPTION

In the present disclosure, it is sought to use brain states that can be determined by EEG signals in applications aimed at improving people's mental performance, such as improving a race car driver's reaction times through training, improving a fighter pilot's combat capability, monitoring a driver's state of alertness, and improving group action execution (eSports, intervention groups). As previously indicated, EEG electrodes are conventionally included in devices worn on users' heads, which have a configuration that makes them poorly suited for field use. Indeed, these devices range from a dedicated rigid or flexible cap covering the entire skull and connected by wires to a remote console, to an elastic band including a sensor dedicated to a limited number of close points, battery powered, and communicating with a data processing unit, such as a terminal or smartphone.

Even a minimalist wristband device is unsuitable for field use. Indeed, the sensor module with its control electronics and battery power is bulky due to the desired autonomy, so that the band cannot be worn under a helmet. In addition, the module is relatively heavy given the size of the battery, so that shocks in the field and sudden head movements may displace the sensor.

In FIG. 1, to be able to use one or more EEG sensors under a helmet with a reduced risk of inadvertent displacement, flexible and thin patch sensors 10 are configured to be adhesively attached to the skin, which communicate EEG data via a radio frequency link (e.g. Bluetooth BT, WiFi, or Ultra-Wide Band UWB), and which are permanently remotely powered by a nearby base station 12 using a conventional technique, such as near field communication NFC or RFID (125 Khz or UHF) power transmission. Thus, the power transmission technique, which in some cases allows data transmission, is used here only for powering the sensors—the EEG data is transmitted via another path, namely a longer range radio frequency link, so that proximity constraints related to NFC or RFID data transmission do not apply.

The base station 12 may be integrated into a helmet 14 and powered by an on-board battery in the base station which is less subject to size constraints, or remotely wired to a suitable location, such as a user's pocket, or a box attached to the outer wall of the helmet.

In the case of a vehicle driver, the base station could also be integrated into a steering wheel, headrest, or ceiling above the driver.

In one embodiment, base station 12 collects the data transmitted by sensors 10 and in turn communicates it to a remote terminal 16, such as a smartphone, via a Bluetooth BT or WiFi wireless link, as represented by solid arrows BT. The terminal may run an application that determines and utilizes the desired brain states.

In some applications, the terminal could be combined with the base station, as in the case of a virtual reality (VR) helmet in which the base station would be connected as a peripheral to the VR helmet's control unit, which control unit is generally connected to a computer via WiFi or a USB cable.

In another embodiment, sensors 10 individually transmit EEG data directly to terminal 16 via their respective radio frequency links, as depicted by dotted arrows BTa to BTc.

In these configurations, base station 12 may need to power sensors up to 1 to 60 cm away from the base station, which implies appropriately sized NFC antennas. In fact, each sensor 10 contemplated here is dedicated to a single EEG point, or a pair of close points—it thus comprises for each point two electrodes, one placed in contact with the corresponding EEG point, on which the voltage is measured (hot point), and the other serving as a reference (cold point). The position of the reference electrode may be arbitrary, but it is preferably located a minimum distance of 2 to 6 centimeters from the hot point. The resulting sensor size thus allows the integration of a relatively large power transmission antennae, so that continuous remote power can be provided by a base station located up to a few tens of centimeters from sensors 10 stuck to the user's skull. When the base station is integrated into a helmet, the distance is less than 10 cm.

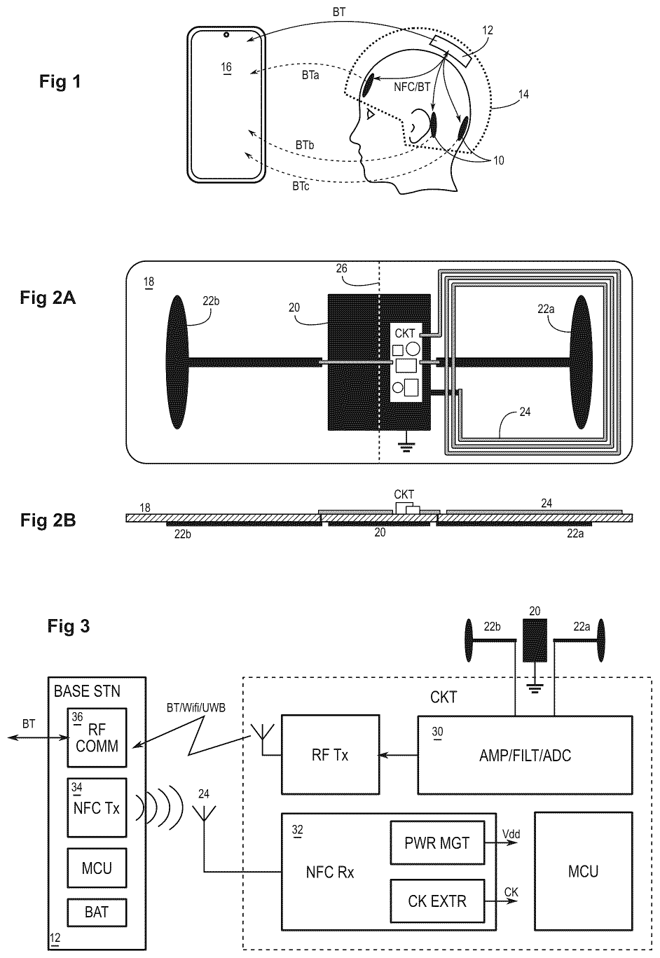

FIGS. 2A and 2B represent a top view and a cross-sectional view of one embodiment of a wireless, remotely powered EEG sensor in the form of an adhesive patch. The sensor shown is adapted to the two forehead or nape points. It comprises a thin, flexible substrate 18, substantially rectangular. The substrate may be suitable for a thin film conductor deposition process. A first face of the substrate, intended to be adhesively attached to the skin, has conductive areas forming a central reference or ground electrode 20 and two symmetrical electrodes 22a, 22b. The distance between electrodes 22a and 22b may be about 5 cm.

The opposite face of the substrate carries, substantially in the center, a control circuit CKT, realized for example using integrated circuits and discrete components fixed by conductive adhesive to conductive tracks formed on the substrate. Electrodes 22a, 22b are connected to circuit CKT by conductors first extending centrally on the first face, then passing through the substrate to join the control circuit on the second face.

A power transmission antenna 24, for example here an NFC type, comprises several turns of a conductor deposited on one of the faces of the substrate, for example the second face. The outer end of the antenna is connected to the control circuit while remaining on the same face, while the inner end passes through the substrate to join the ground electrode 20 on the first face.

As shown, the NFC antenna is made on half of the substrate, and its coils pass as close as possible to the edges of the substrate, around electrode 22a. Control circuit CKT is arranged on the same half of the substrate. This asymmetrical configuration makes it possible, when only one EEG point is to be used (as behind each ear), to cut the sensor in half along a central precut 26, and to use only the right half, including the circuit and antenna 24. Thus, only one type of sensor is manufactured for a multitude of EEG measurement point configurations.

When the sensor is used in its entirety, that is to say with both electrodes 22a and 22b, control circuit CKT individually manages two EEG signals, which may require extra power. If the single antenna 24 is insufficient for power transmission, a similar antenna may be provided on the left half of the sensor, connected in parallel to antenna 24.

FIG. 3 represents a block diagram of the electronic architecture of an EEG signal capture device using a wireless sensor according to FIGS. 2A and 2B. Control circuit CKT comprises an analog subsystem 30 for processing the signals received by electrodes 22a, 22b. The processing may include amplification AMP, filtering FILT and analog-to-digital conversion ADC.

The digital elements of the sensor are controlled by a microcontroller MCU. The required clock and power are provided by a clock extraction unit CK EXTR and a power management unit PWR MGT that are part of an NFC receiver 32 operating on the 13.56 MHz carrier received on antenna 24. NFC receiver 32 may be simplified since it does not have to process any data reception.

Microcontroller MCU processes the digital samples to produce EEG data ready to transmit in data frames according to a standard low power, short range protocol, preferably Bluetooth Low Energy (BLE). Wifi or UWB transmission may also be considered. A corresponding radio frequency transmitter RF Tx receives the data frames and transmits them on an antenna. The transmission may be ensured by an antenna that is small compared to the NFC antenna, also in the form of thin film conductors deposited on the sensor substrate.

Processing of the samples by microcontroller MCU may include various operations, such as data compression by averaging or decimation to reduce the bit rate and thus the power consumption of the transmitter.

Base station 12 includes a simplified NFC transmitter 34, designed to transmit only the carrier used for power transmission.

In the case where the base station 12 is designed to collect the samples transmitted by the sensors, it includes a radio frequency communication unit 36 for receiving the samples and retransmitting them to remote terminal 16. The elements of the base station are controlled by a microcontroller MCU and powered by a battery BAT.

The required transmission range being particularly short for radio frequency in this case (at most 20 cm), the transmission power may be significantly lowered compared to nominal conditions, further reducing sensor power consumption.

In the case where the sensors 10 transmit the samples directly to the terminal 16, the base station 12 may be devoid of communication unit 36 and even microcontroller MCU.

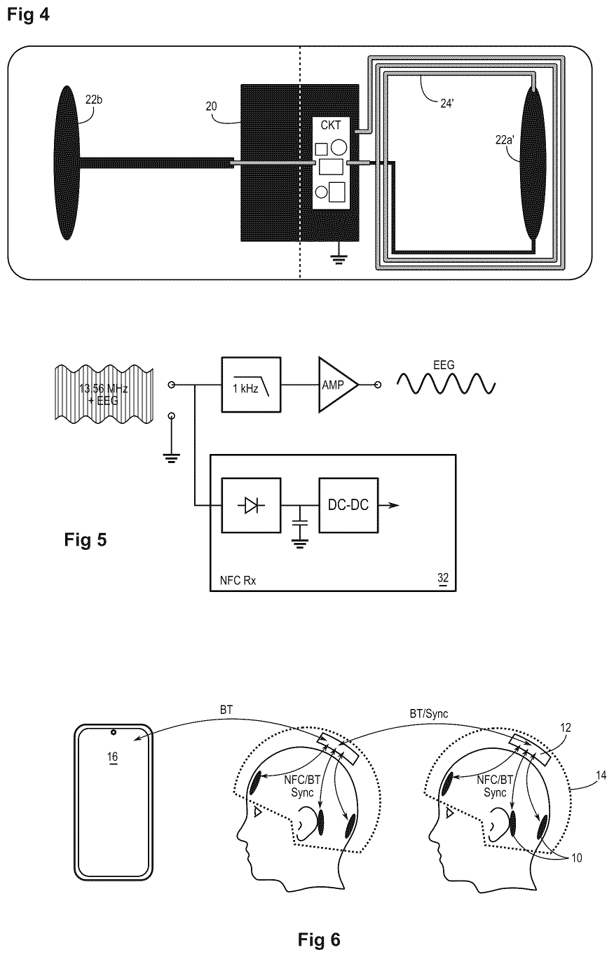

FIG. 4 represents a top view of another embodiment of a wireless EEG sensor. Compared to the sensor of FIG. 2A, the right electrode 22a′ forms a segment of the NFC antenna path 24′. Preferably, electrode 22a′ is on the hot spot side of the antenna, so that the longest part of the antenna track is connected between the electrode and ground, as shown. Thus, at the NFC carrier frequency, the track length between the electrode and ground forms a high impedance, limiting the attenuation of the EEG signal received on electrode 22a′.

FIG. 5 represents an analog subsystem of control circuit CKT, suitable for processing the signal from antenna 24′ of the sensor of FIG. 4. As shown on the left, the EEG signal modulates the amplitude of the 13.56 MHz NFC carrier. This modulated signal feeds two paths, a first one for conventionally extracting the supply current in NFC receiver 32, using a rectifier, filter capacitor, and DC-DC converter. A second path performs low-pass filtering adapted to EEG signals, with a cut-off frequency of 1 kHz, for example, and amplification.

FIG. 6 illustrates an implementation of several wireless sensors distributed over several people. Several users are each equipped with a set of EEG sensors 10 and a corresponding base station 12, preferably integrated into a helmet 14, as in FIG. 1. In the case where the base stations 12 are designed to relay EEG data from the sensors, the radio frequency communication capabilities of the base stations are redundant. Indeed, each base station 12 can be configured to individually transmit its samples to a common remote terminal 16, or, as shown, the base stations can be daisy-chained so that each one transmits its samples to another base station. Then, a last base station in the chain transmits all the collected samples to terminal 16. A base station from a first person can even receive samples transmitted by sensors from another nearby person. Base stations can therefore be designed to dynamically reconfigure themselves based on the best links to optimize power consumption and reduce errors.

In the case where the sensors 10 are configured to individually transmit their EEG data directly to terminal 16, the base stations 12 do not communicate with each other or with the terminal, which simplifies management.

Given the configuration of the sensors 10, each sensor is independent and produces its samples asynchronously. Thus, if the multiple sensors of a device start at different times, or undergo clock drifts because the clocks are provided by different base stations, the temporal correspondence between samples received from different sensors may be lost, which may lead to interpretation errors. Synchronization possibilities for the sensors' EEG signal acquisitions are disclosed below.

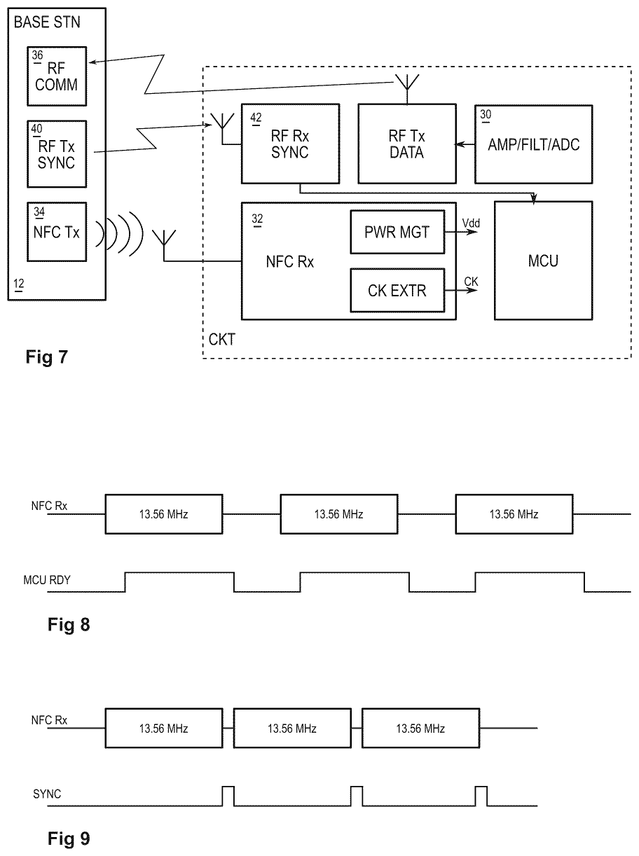

FIG. 7 represents a block diagram of the electronic architecture of an EEG signal capture device implementing a first mode of synchronization of the sensors. Each base station 12 includes a synchronization pulse transmitter 40, using a low bit rate, low power radio frequency band. For example, the 433-434 MHz frequency band typical for very low power radio controls may be used, given the small amount of information to be transmitted (such as a pulse or sequence number).

Each sensor then integrates a corresponding synchronization receiver 42 which extracts the synchronization pulses. The synchronization pulses may interrupt the microcontroller MCU that is then programmed to trigger the acquisition of one or more samples at each synchronization pulse.

In the situation involving multiple people (FIG. 6), each of the base stations would have the ability to synchronize the sensors. In this case, preferably, only one master base station is used for synchronization. The range of the synchronization signals remains sufficiently long to cover multiple people acting as a group. If necessary, the master base station is chosen based on its central position in the group.

As an alternative, a master sensor 10 transmits the synchronization pulses to synchronize all other sensors.

FIG. 8 is a timing diagram illustrating another mode of synchronizing multiple sensors. Base station 12 is configured to periodically suspend the carrier (NFC Rx signal) for a fixed interval. Each sensor 10 thus sees its power cease and resume periodically. The sensor may then be configured so that the first thing achieved when power is restored is acquiring one or more samples. The duration of the power-on interval is long enough that the sensor has time to transmit the samples to the base station. The duty cycle of the power-on sequence may be chosen to optimize the power consumed by the base station.

An MCU RDY signal illustrates the phases where the power is sufficient for the microcontroller. These phases are delayed with respect to the carrier production phases due to the fact that the supply voltage rises each time to a level sufficient to power the microcontroller, and then the microcontroller executes a startup phase before proceeding with the acquisitions. In principle, one can rely on the fact that this delay is constant or varies little.

This synchronization mode applies only between a base station and sensors that are within remote power range. To synchronize the sensors of multiple people, it can be envisaged that the base stations 12 synchronize with each other using the RF communication links used to relay the samples, or dedicated low power RF links (e.g. 433 MHz).

FIG. 9 is a timing diagram illustrating another mode of synchronizing multiple sensors. The base station 12 is configured to periodically interrupt power transmission (NFC Rx signal) at intervals that are sufficiently brief to not interrupt power to the sensors, thanks in particular to charges stored in power supply filter capacitors. The NFC receiver circuit of each sensor 10 may be configured to extract a second clock from these interruption intervals, thus producing a synchronization signal SYNC. The SYNC signal may be used like the radio frequency synchronization pulses in the embodiment of FIG. 7.

As with FIG. 8, this synchronization mode applies only between a base station and sensors within remote power range. To synchronize sensors from multiple people, it may be envisaged that the base stations 12 synchronize with each other using the RF communication links used to relay samples, or dedicated low power RF links (e.g. 433 MHz).

1. A device for capturing electroencephalograms (EEG) signals, the device comprising:
a flexible wireless sensor configured to be adhesively attached at an EEG measurement point of a user's head, and configured to be remotely powered and to transmit, via a first radio frequency link, EEG data corresponding to samples of a measured EEG signal;
a base station located near the sensor, configured to remotely power the sensor; and
a terminal configured to receive transmitted EEG data.

2. The device as claimed in claim 1, wherein the base station is configured to receive EEG data transmitted by the sensor and retransmit the EEG data via a second radio frequency link to the terminal.

3. The device as claimed in claim 1, wherein the terminal is configured to receive EEG data directly from the sensor.

4. The device as claimed in claim 1, wherein the sensor comprises:
a flexible substrate;
on a first face of the substrate, two conductive zones configured to electrically contact the skin and form two electrodes of an EEG signal detector;
a conductive track on one side of the substrate, forming a remote power supply antenna;
a control circuit arranged on a second side of the substrate, connected by conductive tracks of the substrate to the EEG signal detector and to the remote power supply antenna, the control circuit being configured to:
extract its power supply from the signal received by the remote power supply antenna; and
digitize the signal from the EEG signal detector and transmit the corresponding EEG data via the first radio-frequency link.

5. The device as claimed in claim 4, wherein one of the electrode conductor zones is connected to a ground of the control circuit and the other electrode conductor zone forms a segment of the antenna track, the control circuit being configured to extract the power supply and the EEG signal from the antenna signal.

6. The device as claimed in claim 1, wherein the base station is attached to a device worn on the user's head.

7. The device as claimed in claim 1, wherein the base station is configured to synchronize the sampling of EEG signals of the wireless sensor.

8. The device as claimed in claim 1 comprising a plurality of wireless sensors, the wireless sensors cooperating so that a master wireless sensor synchronizes the sampling of EEG signals of all the wireless sensors.

9. The device as claimed in claim 7, wherein the synchronization uses synchronization pulses transmitted by radio frequency in a frequency band distinct from that used for EEG data transmission and remote powering.

10. The device as claimed in claim 7, wherein the base station is configured to suspend the remote power supply at periodic intervals and the wireless sensor is configured to acquire at least one sample of the EEG signal as soon as the power supply to the wireless sensor becomes sufficient after a suspension.

11. The device as claimed in claim 7, wherein the base station is configured to interrupt the remote power supply at periodic intervals brief enough to not interrupt power to the wireless sensor, and the wireless sensor is configured to synchronize EEG signal sampling with the remote power supply interruption intervals.

12. The device as claimed in claim 2, comprising a plurality of wireless sensors distributed over the skulls of a plurality of users, and a plurality of base stations arranged in respective devices carried on the heads of the users, each base station being configured to relay to the remote terminal:
EEG data received from sensors in its remote power supply range,
EEG data received from sensors outside its remote power-supply range, or
EEG data received from another base station.

13. The device as claimed in claim 1, wherein the EEG data is transmitted by the sensor or base station via a WiFi or Bluetooth link.

14. A device for capturing electroencephalograms (EEG) signals, the device comprising:
a flexible wireless sensor configured to be adhesively attached at an EEG measurement point of a user's head, and configured to be remotely powered and to transmit, via a first radio frequency link, EEG data corresponding to samples of a measured EEG signal;
a base station located near the sensor, configured to remotely power the sensor and to synchronize the sampling of EEG signals of the wireless sensor according to one of the two following modes:
(i) suspending the remote power supply at periodic intervals, wherein the wireless sensor is configured to acquire at least one sample of the EEG signal as soon as the power supply to the wireless sensor becomes sufficient after a suspension, and
(ii) interrupting the remote power supply at periodic intervals brief enough to not interrupt power to the wireless sensor, wherein the wireless sensor is configured to synchronize EEG signal sampling with the remote power supply interruption intervals; and
a terminal configured to receive transmitted EEG data.

15. The device as claimed in claim 14, wherein the base station is configured to receive EEG data transmitted by the sensor and retransmit the EEG data via a second radio frequency link to the terminal.

16. The device as claimed in claim 14, wherein the terminal is configured to receive EEG data directly from the sensor.

17. The device as claimed in claim 14, wherein the sensor comprises:
a flexible substrate;
on a first face of the substrate, two conductive zones configured to electrically contact the skin and form two electrodes of an EEG signal detector;
a conductive track on one side of the substrate, forming a remote power supply antenna;
a control circuit arranged on a second side of the substrate, connected by conductive tracks of the substrate to the EEG signal detector and to the remote power supply antenna, the control circuit being configured to:
extract its power supply from the signal received by the remote power supply antenna; and
digitize the signal from the EEG signal detector and transmit the corresponding EEG data via the first radio-frequency link.

18. The device as claimed in claim 17, wherein one of the electrode conductor zones is connected to a ground of the control circuit (CKT) and the other electrode conductor zone forms a segment of the antenna track, the control circuit being configured to extract the power supply and the EEG signal from the antenna signal.

19. The device as claimed in claim 14, wherein the base station is attached to a device worn on the user's head.

20. The device as claimed in claim 15, comprising a plurality of wireless sensors distributed over the skulls of a plurality of users, and a plurality of base stations arranged in respective devices carried on the heads of the users, each base station being configured to relay to the remote terminal:
EEG data received from sensors in its remote power supply range,
EEG data received from sensors outside its remote power-supply range, or
EEG data received from another base station.
